(19) United States
(12) Patent Application Publication
(10) Pub. No.: US 2025/0012770 A1
(43) Pub. Date: Jan. 9, 2025

(54) METHOD AND SYSTEM FOR EVALUATING CARBON SEQUESTRATION OF DUCKWEED FOR RICE-DUCKWEED SYMBIOTIC SYSTEM, AND DEVICE

(71) Applicants: Zhejiang University, Hangzhou City (CN); Hohai University, Nanjing City (CN)

(72) Inventors: Yong HE, Hangzhou City (CN); Yueying WANG, Hangzhou City (CN); Qianjing JIANG, Hangzhou City (CN); Yawei LI, Hangzhou City (CN); Junzeng XU, Hangzhou City (CN)

(21) Appl. No.: 18/423,757

(22) Filed: Jan. 26, 2024

(30) Foreign Application Priority Data
Jul. 5, 2023 (CN) 202310815779.1

(51) Int. Cl. G01N 33/00; G01N 21/27

(52) U.S. Cl. CPC G01N 33/0098; G01N 21/27

(57) ABSTRACT

The present disclosure provides a method and system for evaluating carbon sequestration of duckweed for a rice-duckweed symbiotic system, and a device. The method includes: after rice harvesting, collecting duckweed information in a rice-duckweed symbiotic system using spectrum scanning and laser-point cloud techniques, and determining a duckweed volume based on the duckweed information; determining a duckweed biomass in the rice-duckweed symbiotic system based on the duckweed volume; and estimating carbon sequestration of duckweed from the duckweed biomass using an artificial neural network algorithm, or estimating the carbon sequestration of duckweed from the duckweed biomass by a duckweed carbon sequestration estimation formula. The present disclosure may allow for flexible estimation of carbon sequestration of duckweed based on information such as agronomic management on rice, soil, and climate, and provide a good technical support for the management and application of duckweed in paddy fields.

### CROSS REFERENCE TO RELATED APPLICATION

This patent application claims the benefit and priority of Chinese Patent Application No. 202310815779.1, filed with the China National Intellectual Property Administration on Jul. 5, 2023, the disclosure of which is incorporated by reference herein in its entirety as part of the present application.

### TECHNICAL FIELD

The present disclosure relates to the technical fields of agricultural production and ecological environment, and in particular, to a method and system for evaluating carbon sequestration of duckweed for a rice-duckweed symbiotic system, and a device.

### BACKGROUND

Duckweed is a small aquatic phytoplankton common in shallow-water environments such as ponds, paddy fields, and puddles. In the early years, the duckweed was commonly regarded as a harmful species resulting in a reduction in production of a paddy field and was usually removed by salvaging, pesticide spraying, and the like. As people are paying more attention to the environmental pollution problem, the benefit of rice-duckweed symbiosis for the health of the environmental ecology has been found. Studies found that the rice-duckweed symbiosis can reduce ammonia volatilization and greenhouse gas emission from a paddy field, inhibit the occurrence and propagation of rice diseases and insect pests, and enhance the stability of the ecological environment of the paddy field.

Unlike terrestrial plants, the duckweed does not need to strongly rely on the root system and can absorb nutrient substances such as nitrogen and phosphorus in water directly through a plant tissue surrounded by a liquid. The duckweed absorbs and stores nutrients during growth. After rice harvesting, the duckweed is air-dried and returned to farmland soil. On the one hand, exogenous organic matter input is increased to increase a soil organic carbon content and retard agricultural non-point source pollution; and on the other hand, nitrogen and phosphorus elements contained in the duckweed can increase a primary mineralization rate of nitrogen and phosphorus in soil, reduce soil leaching and runoff losses, and reduce a soil organic carbon decomposition rate. Therefore, the duckweed can absorb and fix CO₂ in the atmosphere and nitrogen and phosphorus in water in the processes of alternation of wetting and drying of soil and “growth-death-decomposition” of the duckweed itself, thereby increasing carbon sequestration of soil and counteracting partial carbon budget imbalance of a paddy field.

Carbon sequestration is a process, activity, and mechanism of removing CO₂ from air, and how to immediately and effectively estimate carbon sequestration of an area poses a challenge for the scientific community. An inventory method is suitable for widely distributed ecological systems such as forests and grasslands with rich data. An eddy covariance method is mainly based on the principles of micrometeorology, by which it is hard to accurately estimate the carbon budget of the agricultural ecological system. A simulation method based on an ecological system process model universally does not take into account or simply takes into account the influence of ecological system management (such as soil management and agricultural irrigation) on carbon cycling. An atmospheric retrieval method is suitable for large-scale targets, and since a target area of a paddy field is too small, a retrieval result has great uncertainty.

Therefore, there is current no rapid, accurate, and flexible method for estimating carbon sequestration of duckweed (i.e., increased carbon sequestration of soil in the decomposition process of duckweed returned to the field), thus realizing partial compensation of ecological benefits and facilitating the development of the carbon sequestration economy of a paddy field.

### SUMMARY

An objective of the present disclosure is to provide a method and system for evaluating carbon sequestration of duckweed for a rice-duckweed symbiotic system, and a device that may allow for flexible estimation of carbon sequestration of duckweed based on information such as agronomic management on rice, soil, and climate, and provide a good technical support for the management and application of duckweed in paddy fields.

To achieve the above objective, the present disclosure provides the following technical solutions.

In a first aspect, the present disclosure provides a method for evaluating carbon sequestration of duckweed for a rice-duckweed symbiotic system, including:
after rice harvesting, collecting duckweed information in a rice-duckweed symbiotic system using spectrum scanning and laser-point cloud techniques, and determining a duckweed volume based on the duckweed information;
determining a duckweed biomass in the rice-duckweed symbiotic system based on the duckweed volume; and
estimating carbon sequestration of duckweed from the duckweed biomass using an artificial neural network algorithm, or estimating the carbon sequestration of duckweed from the duckweed biomass by a duckweed carbon sequestration estimation formula.

In a second aspect, the present disclosure provides a system for evaluating carbon sequestration of duckweed for a rice-duckweed symbiotic system, including:
a duckweed information and duckweed volume determining module configured to, after rice harvesting, collect duckweed information in a rice-duckweed symbiotic system using spectrum scanning and laser-point cloud techniques, and determine a duckweed volume based on the duckweed information;
a duckweed biomass calculating module configured to determine a duckweed biomass in the rice-duckweed symbiotic system based on the duckweed volume; and
a duckweed carbon sequestration estimating module configured to estimate carbon sequestration of duckweed from the duckweed biomass using an artificial neural network algorithm, or estimate the carbon sequestration of duckweed from the duckweed biomass by a duckweed carbon sequestration estimation formula.

In a third aspect, the present disclosure provides an electronic device, including a memory and a processor, where the memory is configured to store a computer program, and the processor runs the computer program to cause the electronic device to perform the method for evaluating carbon sequestration of duckweed for a rice-duckweed symbiotic system according to the first aspect.

According to specific embodiments provided in the present disclosure, the present disclosure has the following technical effects:

The present disclosure allows for estimation of carbon sequestration of duckweed using an artificial neural network algorithm or by a duckweed carbon sequestration estimation formula on the basis of spectrum scanning and laser-point cloud techniques. The present disclosure solves the problems of inapplicability of existing carbon sequestration calculation methods in the rice-duckweed symbiotic system, and high time cost, poor mobility, and the like of an agricultural system model, thereby making it possible to effectively estimate carbon sequestration of duckweed in a regional paddy field and further providing a good technical support for the management and application of duckweed in the paddy field.

### BRIEF DESCRIPTION OF THE DRAWINGS

To describe the technical solutions in embodiments of the present disclosure or in the prior art more clearly, the accompanying drawings required in the embodiments are briefly described below. Apparently, the accompanying drawings in the following description show merely some embodiments of the present disclosure, and other drawings can be derived from these accompanying drawings by those of ordinary skill in the art without creative efforts.

FIG. 1 is a flowchart of a method for evaluating carbon sequestration of duckweed for a rice-duckweed symbiotic system provided in an embodiment of the present disclosure; and

FIG. 2 is a structural block diagram of a system for evaluating carbon sequestration of duckweed for a rice-duckweed symbiotic system provided in an embodiment of the present disclosure.

### DETAILED DESCRIPTION OF THE EMBODIMENTS

The technical solutions of the embodiments of the present disclosure are clearly and completely described below with reference to the drawings in the embodiments of the present disclosure. Apparently, the described embodiments are merely a part rather than all of the embodiments of the present disclosure. All other embodiments derived from the embodiments in the present disclosure by a person of ordinary skill in the art without creative efforts shall fall within the protection scope of the present disclosure.

In order to make the above objective, features, and advantages of the present disclosure clearer and more comprehensible, the present disclosure will be further described in detail below in combination with accompanying drawings and particular implementation modes.

### Example 1

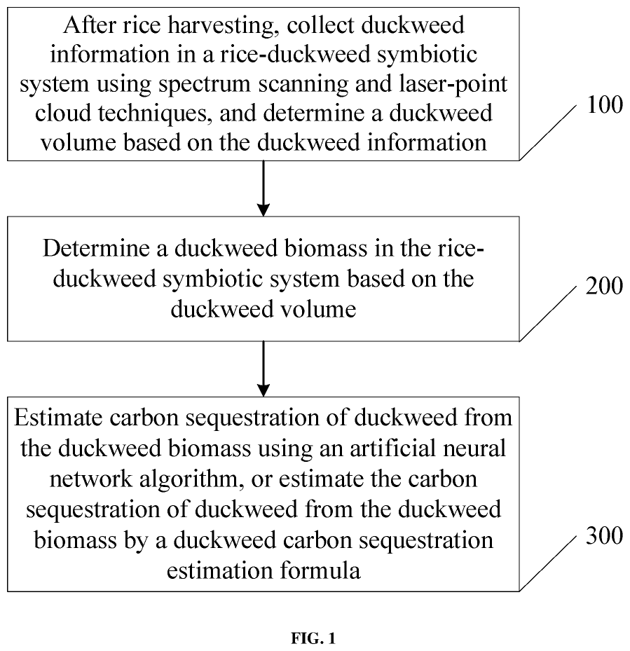

As shown in FIG. 1, a method for evaluating carbon sequestration of duckweed for a rice-duckweed symbiotic system provided in this example includes the following steps.

Step 100: after rice harvesting, duckweed information in a rice-duckweed symbiotic system is collected using spectrum scanning and laser-point cloud techniques, and a duckweed volume is determined based on the duckweed information.

In this example, determining a duckweed volume based on the duckweed information specifically includes:

reconstruct a closed point cloud surface based on the duckweed information using an image and point cloud based three-dimensional reconstruction technique, and calculate a volume of the closed point cloud surface, namely the duckweed volume (V) using a Monte Carlo algorithm.

Step 200: a duckweed biomass in the rice-duckweed symbiotic system is determined based on the duckweed volume.

In this example, step 200 specifically includes:
calculate the duckweed biomass in the rice-duckweed symbiotic system based on a self-defining function for a duckweed biomass, the duckweed volume, and a conversion coefficient for a duckweed volume and a duckweed biomass.

In this example, the self-defining function for a duckweed biomass is solved using python to obtain a numerical value. The self-defining function for a duckweed biomass is as follows:

$$B = ln \vert {V}^{ζ} - {T}_{i} \vert ;$$

where B represents the duckweed biomass, in g/m³; V represents the duckweed volume, in m³; ζ represents the conversion coefficient for a duckweed volume and a duckweed biomass; Tᵢ represents a constant parameter related to a duckweed variety; i represents a duckweed variety; and i=1, 2, 3, 4 represent diamond-shaped duckweed, ordinary duckweed, small duckweed, and root-derived duckweed, respectively.

Step 300: carbon sequestration of duckweed is estimated from the duckweed biomass using an artificial neural network algorithm, or the carbon sequestration of duckweed is estimated from the duckweed biomass by a duckweed carbon sequestration estimation formula.

In this example, before step 300, the method further includes:
1) correct the duckweed biomass with an initial placement density of duckweed and water property parameters, where a formula for correcting the duckweed biomass is as follows:

$${B}_{c} = B \cdot \frac{{ρ}_{d} \cdot \vert 7 - pH \vert}{\vert {ρ}_{r} - 1.1 \vert} - {\vert \frac{{N}_{w} + {N}_{T}}{{N}_{w} - {N}_{T}} \vert}^{\sqrt{DO \cdot d}} ;$$

where B_{c} represents a corrected duckweed biomass, in g/m³; B represents the duckweed biomass, in g/m³; ρ_{d} represents the initial placement density of duckweed, in plants/m³; ρᵣ represents a rice planting density, in ten thousand holes; pH represents a pH value of paddy water; d represents a depth of the paddy water, in m; N_{w} represents a nitrogen concentration of the paddy water, in kg·L⁻¹; DO represents a dissolved oxygen content of the paddy water, in mg·L⁻¹; and N_{T} represents a nitrogen application concentration during rice planting, in kg·L⁻¹.

In this example, estimating carbon sequestration of duckweed from the duckweed biomass using an artificial neural network algorithm specifically includes:
firstly, construct a duckweed carbon sequestration estimation model with a plurality of pieces of historical data by the artificial neural network algorithm; and second, estimate carbon sequestration of duckweed at the present stage, namely the increased organic carbon content of soil after the duckweed is returned to the soil, based on the corrected duckweed biomass using the duckweed carbon sequestration estimation model.

The historical data includes input data and corresponding label data. A soil organic carbon content, a duckweed biomass, an organic carbon content of duckweed, a microbial quantity in soil, a depth of ploughing, a temperature and a humidity, a total nitrogen content, and a C/N ratio are input data, and the carbon sequestration of duckweed is the label data.

In this example, the duckweed carbon sequestration estimation formula is as follows:

$$CS = [ log \frac{SOC}{{B}_{C} \cdot OC} \cdot {( {εM}_{a} )}^{H} \cdot ( 0.424 \cdot {T}^{0.499} + 0.575 \cdot {T}^{0.023} ) ] - ( \frac{1 + SOC \cdot {η}_{j}^{{d}_{S} \cdot H}}{\sqrt{12.4 - \frac{{T}_{S}}{{T}_{a}}}} ) + ln ( \frac{{H}^{2}}{2} \cdot \frac{3}{11} ) \cdot Exp ( \frac{{TN}_{S} \cdot {P}_{d}}{{TN}_{d} \cdot {P}_{S}} ) ;$$

where CS represents carbon sequestration amount; in PgC·yr⁻¹; SOC represents a soil organic carbon content (e.g., a soil organic carbon content obtained using a total organic carbon analyzer), in g·kg⁻¹; B_{c} represents the corrected duckweed biomass, in g/m³; OC represents an organic carbon content of duckweed, in %; ε represents the conversion coefficient; Mₐ represents of a microbial quantity in soil, in CFU·g⁻¹; T represents a duckweed decomposition time, in day; ηⱼ represents a carbon emission factor in a different ploughing mode; j=1, 2, 3 represent ploughing, rotary tillage, and no-tillage with straw mulch, respectively; dₛ represents of depth of ploughing, in m; H represents an average humidity at 0-20 cm of soil, in % rh; Tₛ represents an average temperature at 0-20 cm of soil, in ° C.; Tₐ represents an air temperature, in ° C.; TNₛ represents a total nitrogen content at 0-20 cm of soil, in %; TN_{d} represents a total nitrogen content of duckweed, in %; P_{d} represents a C/N ratio in duckweed; and Pₛ represents a C/N ratio in soil.

In this example, the organic carbon content of duckweed OC and the total nitrogen content (Total N) of duckweed are measured using the total organic carbon analyzer and ultraviolet spectrophotometry, and then the carbon-nitrogen ratio (C/N) of duckweed is calculated. The total nitrogen content (Total N) of duckweed is TN_{d}; the carbon-nitrogen ratio of duckweed is P_{d}; and TNₛ and Pₛ are parameters of soil measured separately.

Correspondingly, the carbon sequestration of duckweed per unit biomass is as follows:

$${CS}_{d} = \frac{CS}{Bc} .$$

### Example 2

In order to perform the corresponding method in Example 1 to realize the corresponding features and technical effects, a system for evaluating carbon sequestration of duckweed for a rice-duckweed symbiotic system is provided below.

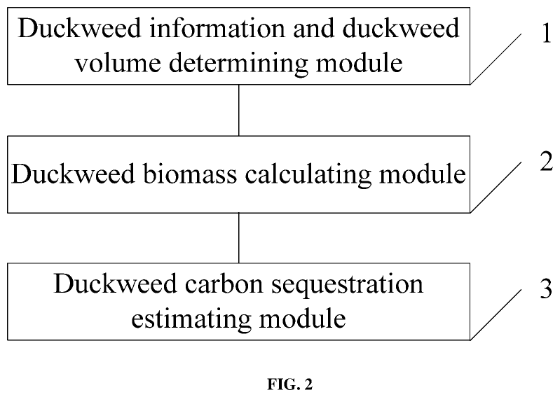

As shown in FIG. 2, the system for evaluating carbon sequestration of duckweed for a rice-duckweed symbiotic system provided in this example includes:
a duckweed information and duckweed volume determining module 1 configured to, after rice harvesting, collect duckweed information in a rice-duckweed symbiotic system using spectrum scanning and laser-point cloud techniques, and determine a duckweed volume based on the duckweed information;
a duckweed biomass calculating module 2 configured to determine a duckweed biomass in the rice-duckweed symbiotic system based on the duckweed volume; and
a duckweed carbon sequestration estimating module 3 configured to estimate carbon sequestration of duckweed from the duckweed biomass using an artificial neural network algorithm, or estimate the carbon sequestration of duckweed from the duckweed biomass by a duckweed carbon sequestration estimation formula.

### Example 3

An example of the present disclosure provides an electronic device, including a memory and a processor, where the memory is configured to store a computer program, and the processor runs the computer program to cause the electronic device to perform the method for evaluating carbon sequestration of duckweed for a rice-duckweed symbiotic system of Example 1.

Alternatively, the electronic device described above may be a server.

In addition, an embodiment of the present disclosure further provides a computer-readable storage medium storing a computer program, and the computer program, when executed by a processor, causes implementing the method for evaluating carbon sequestration of duckweed for a rice-duckweed symbiotic system of Example 1.

Each embodiment in the description is described in a progressive mode, each embodiment focuses on differences from other embodiments, and references can be made to each other for the same and similar parts between embodiments. Since the system disclosed in an embodiment corresponds to the method disclosed in an embodiment, the description is relatively simple, and for related contents, references can be made to the description of the method.

Particular examples are used herein for illustration of principles and implementation modes of the present disclosure. The descriptions of the above embodiments are merely used for assisting in understanding the method of the present disclosure and its core ideas. In addition, those of ordinary skill in the art can make various modifications in terms of particular implementation modes and the scope of application in accordance with the ideas of the present disclosure. In conclusion, the content of the description shall not be construed as limitations to the present disclosure.

What is claimed is:

1. A method for evaluating carbon sequestration of duckweed for a rice-duckweed symbiotic system, comprising:
after rice harvesting, collecting duckweed information in a rice-duckweed symbiotic system using spectrum scanning and laser-point cloud techniques, and determining a duckweed volume based on the duckweed information;
determining a duckweed biomass in the rice-duckweed symbiotic system based on the duckweed volume; and
estimating carbon sequestration of duckweed from the duckweed biomass using an artificial neural network algorithm, or estimating the carbon sequestration of duckweed from the duckweed biomass by a duckweed carbon sequestration estimation formula.

2. The method for evaluating carbon sequestration of duckweed for a rice-duckweed symbiotic system according to claim 1, wherein the determining a duckweed volume based on the duckweed information specifically comprises:
reconstructing a closed point cloud surface based on the duckweed information using an image and point cloud based three-dimensional reconstruction technique, and calculating a volume of the closed point cloud surface using a Monte Carlo algorithm, wherein the volume of the closed point cloud surface is the duckweed volume.

3. The method for evaluating carbon sequestration of duckweed for a rice-duckweed symbiotic system according to claim 1, wherein the determining a duckweed biomass in the rice-duckweed symbiotic system based on the duckweed volume specifically comprises:
calculating the duckweed biomass in the rice-duckweed symbiotic system based on a self-defining function for a duckweed biomass, the duckweed volume, and a conversion coefficient for a duckweed volume and a duckweed biomass.

4. The method for evaluating carbon sequestration of duckweed for a rice-duckweed symbiotic system according to claim 3, wherein the self-defining function for a duckweed biomass is as follows:
$$B = ln \vert {V}^{ζ} - {T}_{i} \vert ;$$
wherein B represents the duckweed biomass, in g/m³; V represents the duckweed volume, in m³; ζ represents the conversion coefficient for a duckweed volume and a duckweed biomass; Tᵢ represents a constant parameter related to a duckweed variety; i represents a duckweed variety; and i=1, 2, 3, 4 represent diamond-shaped duckweed, ordinary duckweed, small duckweed, and root-derived duckweed, respectively.

5. The method for evaluating carbon sequestration of duckweed for a rice-duckweed symbiotic system according to claim 1, before the estimating carbon sequestration of duckweed from the duckweed biomass using an artificial neural network algorithm, or the estimating the carbon sequestration of duckweed from the duckweed biomass by a duckweed carbon sequestration estimation formula, further comprising:
correcting the duckweed biomass with an initial placement density of duckweed and water property parameters.

6. The method for evaluating carbon sequestration of duckweed for a rice-duckweed symbiotic system according to claim 5, wherein a formula for correcting the duckweed biomass is as follows:
$${B}_{c} = B \cdot \frac{{ρ}_{d} \cdot \vert 7 - pH \vert}{\vert {ρ}_{r} - 1.1 \vert} - {\vert \frac{{N}_{w} + {N}_{T}}{{N}_{w} - {N}_{T}} \vert}^{\sqrt{DO \cdot d}} ;$$
wherein B_{c} represents a corrected duckweed biomass, in g/m³; B represents the duckweed biomass, in g/m³; ρ_{d} represents the initial placement density of duckweed, in plants/m³; ρᵣ represents a rice planting density, in ten thousand holes; pH represents a pH value of paddy water; d represents a depth of the paddy water, in m; N_{w} represents a nitrogen concentration of the paddy water, in kg·L⁻¹; DO represents a dissolved oxygen content of the paddy water, in mg·L⁻¹; and N_{T} represents a nitrogen application concentration during rice planting, in kg·L⁻¹.

7. The method for evaluating carbon sequestration of duckweed for a rice-duckweed symbiotic system according to claim 5, wherein the duckweed carbon sequestration estimation formula is as follows:
$$CS = [ log \frac{SOC}{{B}_{C} \cdot OC} \cdot {( {εM}_{a} )}^{H} \cdot ( 0.424 \cdot {T}^{0.499} + 0.575 \cdot {T}^{0.023} ) ] - ( \frac{1 + SOC \cdot {η}_{j}^{{d}_{S} \cdot H}}{\sqrt{12.4 - \frac{{T}_{S}}{{T}_{a}}}} ) + ln ( \frac{{H}^{2}}{2} \cdot \frac{3}{11} ) \cdot Exp ( \frac{{TN}_{S} \cdot {P}_{d}}{{TN}_{d} \cdot {P}_{S}} ) ;$$
wherein CS represents carbon sequestration amount; in PgC·yr⁻¹; SOC represents a soil organic carbon content, in g·kg⁻¹; B_{c} represents the corrected duckweed biomass, in g/m³; OC represents an organic carbon content of duckweed, in %; E represents the conversion coefficient; Mₐ represents of a microbial quantity in soil, in CFU·g⁻¹; T represents a duckweed decomposition time, in day; ηⱼ represents a carbon emission factor in a different ploughing mode; j=1, 2, 3 represent ploughing, rotary tillage, and no-tillage with straw mulch, respectively; dₛ represents of depth of ploughing, in m; H represents an average humidity at 0-20 cm of soil, in % rh; Tₛ represents an average temperature at 0-20 cm of soil, in ° C.; Tₐ represents an air temperature, in ° C.; TNₛ represents a total nitrogen content at 0-20 cm of soil, in %; TN_{d} represents a total nitrogen content of duckweed, in %; P_{d} represents a C/N ratio in duckweed; and Pₛ represents a C/N ratio in soil.

8. A system for evaluating carbon sequestration of duckweed for a rice-duckweed symbiotic system, comprising:
a duckweed information and duckweed volume determining circuit configured to, after rice harvesting, collect duckweed information in a rice-duckweed symbiotic system using spectrum scanning and laser-point cloud techniques, and determine a duckweed volume based on the duckweed information;
a duckweed biomass calculating circuit configured to determine a duckweed biomass in the rice-duckweed symbiotic system based on the duckweed volume; and
a duckweed carbon sequestration estimating circuit configured to estimate carbon sequestration of duckweed from the duckweed biomass using an artificial neural network algorithm, or estimate the carbon sequestration of duckweed from the duckweed biomass by a duckweed carbon sequestration estimation formula.

9. An electronic device, comprising a memory and a processor, wherein the memory is configured to store a computer program, and the processor runs the computer program to cause the electronic device to perform operations to:
after rice harvesting, collect duckweed information in a rice-duckweed symbiotic system using spectrum scanning and laser-point cloud techniques, and determining a duckweed volume based on the duckweed information;
determine a duckweed biomass in the rice-duckweed symbiotic system based on the duckweed volume; and
estimate carbon sequestration of duckweed from the duckweed biomass using an artificial neural network algorithm, or estimate the carbon sequestration of duckweed from the duckweed biomass by a duckweed carbon sequestration estimation formula.

10. The electronic device of claim 9, wherein the operation to determine a duckweed volume based on the duckweed information, comprises to:
reconstruct a closed point cloud surface based on the duckweed information using an image and point cloud based three-dimensional reconstruction technique, and calculating a volume of the closed point cloud surface using a Monte Carlo algorithm, wherein the volume of the closed point cloud surface is the duckweed volume.

11. The electronic device of claim 9, wherein the operation to determine a duckweed biomass in the rice-duckweed symbiotic system based on the duckweed volume, comprises to:
calculate the duckweed biomass in the rice-duckweed symbiotic system based on a self-defining function for a duckweed biomass, the duckweed volume, and a conversion coefficient for a duckweed volume and a duckweed biomass.

12. The electronic device of claim 11, wherein the self-defining function operation for a duckweed biomass is computed based on:
$$B = ln \vert {V}^{ζ} - {T}_{i} \vert ;$$
wherein B represents the duckweed biomass, in g/m³; V represents the duckweed volume, in m³; ζ represents the conversion coefficient for a duckweed volume and a duckweed biomass; Tᵢ represents a constant parameter related to a duckweed variety; i represents a duckweed variety; and i=1, 2, 3, 4 represent diamond-shaped duckweed, ordinary duckweed, small duckweed, and root-derived duckweed, respectively.

13. The electronic device of claim 9, wherein before the operation to estimate carbon sequestration of duckweed from the duckweed biomass using an artificial neural network algorithm, or the operation to estimate the carbon sequestration of duckweed from the duckweed biomass by a duckweed carbon sequestration estimation formula, the operations further comprise to:
correct the duckweed biomass with an initial placement density of duckweed and water property parameters.

14. The electronic device of claim 13, wherein a formula used to correct the duckweed biomass is computed based on:
$${B}_{c} = B \cdot \frac{{ρ}_{d} \cdot \vert 7 - pH \vert}{\vert {ρ}_{r} - 1.1 \vert} - {\vert \frac{{N}_{w} + {N}_{T}}{{N}_{w} - {N}_{T}} \vert}^{\sqrt{DO \cdot d}} ;$$
wherein B_{c} represents a corrected duckweed biomass, in g/m³; B represents the duckweed biomass, in g/m³; ρ_{d} represents the initial placement density of duckweed, in plants/m³; ρᵣ represents a rice planting density, in ten thousand holes; pH represents a pH value of paddy water; d represents a depth of the paddy water, in m; N_{w} represents a nitrogen concentration of the paddy water, in kg·L⁻¹; DO represents a dissolved oxygen content of the paddy water, in mg·L⁻¹; and N_{T} represents a nitrogen application concentration during rice planting, in kg·L⁻¹.

15. The electronic device of claim 13, wherein the duckweed carbon sequestration estimation formula is computed based on:
$$CS = [ log \frac{SOC}{{B}_{C} \cdot OC} \cdot {( {εM}_{a} )}^{H} \cdot ( 0.424 \cdot {T}^{0.499} + 0.575 \cdot {T}^{0.023} ) ] - ( \frac{1 + SOC \cdot {η}_{j}^{{d}_{S} \cdot H}}{\sqrt{12.4 - \frac{{T}_{S}}{{T}_{a}}}} ) + ln ( \frac{{H}^{2}}{2} \cdot \frac{3}{11} ) \cdot Exp ( \frac{{TN}_{S} \cdot {P}_{d}}{{TN}_{d} \cdot {P}_{S}} ) ;$$
wherein CS represents carbon sequestration amount; in PgC·yr⁻¹; SOC represents a soil organic carbon content, in g·kg⁻¹; B_{c} represents the corrected duckweed biomass, in g/m³; OC represents an organic carbon content of duckweed, in %; E represents the conversion coefficient; Mₐ represents of a microbial quantity in soil, in CFU·g⁻¹; T represents a duckweed decomposition time, in day; ηⱼ represents a carbon emission factor in a different ploughing mode; j=1, 2, 3 represent ploughing, rotary tillage, and no-tillage with straw mulch, respectively; dₛ represents of depth of ploughing, in m; H represents an average humidity at 0-20 cm of soil, in % rh; Tₛ represents an average temperature at 0-20 cm of soil, in ° C.; Tₐ represents an air temperature, in ° C.; TNₛ represents a total nitrogen content at 0-20 cm of soil, in %; TN_{d} represents a total nitrogen content of duckweed, in %; P_{d} represents a C/N ratio in duckweed; and Pₛ represents a C/N ratio in soil.
